(19) United States
(12) Patent Application Publication
(10) Pub. No.: US 2018/0214679 A1
(43) Pub. Date: Aug. 2, 2018

(54) IDENTIFICATION SYSTEM FOR INJECTABLE ACCESS PORTS

(71) Applicant: pfm Medical, Inc., Carlsbad, CA (US)

(72) Inventor: Jiaye Jho, Carlsbad, CA (US)

(21) Appl. No.: 15/421,756

(22) Filed: Feb. 1, 2017

(51) Int. Cl. A61M 39/02

(52) U.S. Cl. CPC A61M 39/0208; A61M 2039/0238; A61M 2039/0205

(57) ABSTRACT

A non-power injectable vascular access port configured to be implanted subcutaneously having a housing; a septum affixed to the housing; an internal reservoir collectively defined by the septum and the housing; an outlet configured to be in fluid communication with the internal reservoir; and one or more markers discernable following subcutaneous implantation of the access port and configured to identify the vascular access port as suitable for a non-power injection fluid flow rate and unsuitable for a power injection fluid flow rate. Related systems and methods of use are described.

### FIELD

The present technology relates generally to vascular access port. More particularly, the present technology relates to vascular access port identification systems.

### BACKGROUND

Intravenous (IV) therapy involves the delivery of liquid substances directly into a blood vessel. Such therapy may be intermittent or may be continuous. During therapy, a fluid conduit must be established into the vascular system of the patient and maintained. A wide variety of medical procedures require infusion of a fluid into a patient. When repeated infusions are required, a peripheral IV line may be used such that prolonged therapy and multiple doses may be provided without inserting a needle into the bloodstream each dose. A catheter can be inserted through the patient's skin into a sealed engagement with a vessel. A body or hub in sealed communication with the axial passage of the catheter can be engaged on an end of the catheter and remain outside the patient's body, usually on the skin surface. In this configuration, the hub can be connected to a syringe or an intravenous infusion line to communication fluid to the bloodstream of the patient, or capped when not in use. The hub and engaged catheter allows for multiple treatments with the same line.

Many patients, however, require a more direct route to the central blood vessels for provision of medication, treatments, and injections employed during X-ray and other imaging. Conventionally, a central venous line provides access for this purpose such that the catheter is inserted into a subclavian, internal jugular, or (less commonly) a femoral vein and advanced toward the heart until it reaches the inferior vena cava, superior vena cava or right atrium. Because all of these veins are larger than peripheral veins, central lines can be employed to deliver a much higher volume of fluid and can also have multiple lumens feeding the central line.

Implantable ports are a type of venous line that does not employ an external connector positioned outside the patient's body. Instead, implantable ports have a small reservoir covered with a flexible cover and the entire device is implanted under the skin of the patient. An outlet of the reservoir communicates with an internal blood vessel such as a vein via a catheter having a lumen. Once implanted, medication may be administered to the patient by communicating a small Huber needle through the patient's skin, piercing the septum or flexible cover of the port such that medication can be injected directly into the reservoir under the flexible cover provided by the septum. When the needle is withdrawn, the reservoir cover reseals. The septum can be penetrated repeatedly in this manner such that the port may be left in the patient's body for years to help avoid infection by leaving the skin barrier intact. Implantable ports improve patient comfort because fewer needle sticks and the lack of exterior mounted components.

### SUMMARY

In some aspects there are provided systems, devices and methods for using injectable vascular access ports.

In some aspects, there is provided a non-power injectable vascular access port configured to be implanted subcutaneously. The port includes a housing; a septum affixed to the housing; an internal reservoir collectively defined by the septum and the housing; an outlet configured to be in fluid communication with the internal reservoir; and one or more markers discernable following subcutaneous implantation of the access port and configured to identify the vascular access port as suitable for a non-power injection fluid flow rate and unsuitable for a power injection fluid flow rate.

The non-power injection fluid flow rate can be no more than about 1 mL/second through the port. The port can be configured to withstand an internal pressure of no more than about 35 psi at a temperature of about 37° C. The one or more markers can be formed of a material that is discernable on a CT scan, X-ray, or fluoroscope relative to surrounding tissues as well as materials of the port. The material can be nitinol, tungsten, titanium, stainless steel, aluminum, copper, tin, nickel, non-ferrous metal, high density ceramic, gadolinium oxysulfide, silicone nitride, zirconium, zirconium oxide, X-ray excitable polymer, PTFE, and/or PTFE impregnated with a non-ferrous metal. The one or more markers can be an ink printed on a surface of the port. The one or more markers can be a structural element coupled to one or more regions of the access port. The one or more markers can be two-dimensional or three-dimensional. The one or more markers can be an elongated wire, ribbon, thread, fiber, or columnar element. The one or more markers can be a mesh, fabric, coating, or other generally planar element. The one or more markers can be formed from a solid piece of metal material. The one or more markers can be penetrable by a cannula or syringe needle inserted through the septum. The one or more markers can be arranged in the shape of a non-alphanumeric symbol and/or shape. The one or more markers can be a non-letter symbol. The non-letter symbol can be an exclamation point, a lightning bolt with an X through it, or a triangle with an X through it. The one or more markers can be an alphanumeric symbol, word or phrase.

The above-noted aspects and features may be implemented in systems, apparatus, methods depending on the desired configuration. The details of one or more variations of the subject matter described herein are set forth in the accompanying drawings and the description below. Features and advantages of the subject matter described herein will be apparent from the description and drawings, and from the claims.

### BRIEF DESCRIPTION OF THE DRAWINGS

These and other aspects will now be described in detail with reference to the following drawings. Generally speaking the figures are not to scale in absolute terms or comparatively, but are intended to be illustrative. Also, relative placement of features and elements may be modified for the purpose of illustrative clarity.

FIG. 1A illustrates a perspective view of an implementation of a vascular access port;

FIG. 1B illustrates a cross-sectional side view of the vascular access port of FIG. 1A;

FIG. 1C illustrates a detailed view of FIG. 1B taken along circle C-C;

FIG. 1D illustrates an exploded, partial view of a vascular access port;

FIGS. 2A-2B illustrate side views of an implementation of a vascular access port;

FIG. 2C illustrates a top plan view of the vascular access port of FIG. 2A;

FIGS. 3A-3D illustrate various implementations of a non-power injection marker for a vascular access port;

FIGS. 4A-4B illustrate side views of an implementation of a vascular access port;

FIG. 4C illustrates a top plan view of the vascular access port of FIG. 4A;

FIGS. 5A-5D illustrate various implementations of a power injection marker on a connecting element of a vascular access port;

FIGS. 6A-6D illustrate various implementations of a non-power injection marker on a connecting element of a vascular access port.

It should be appreciated that the drawings are for example only and are not meant to be to scale. It is to be understood that devices described herein may include features not necessarily depicted in each figure.

### DETAILED DESCRIPTION

Subcutaneous vascular access ports for introducing a fluid into the vasculature of a patient provide a convenient way to repeatedly deliver medicaments. Some implantable vascular access ports are configured to withstand what is known in the art as a power injection. Power injections involve large volumes of liquid injected into the reservoir of the implanted port under high pressure and over a short time without resulting in rupture or malfunction of the various components of the system. For example, vascular imaging technologies may require use of contrast media that is injected into the patient. Computed tomography (CT) is an imaging technology that uses a contrast media employed to noninvasively evaluate and assess a vascular system (i.e. CT angiography or CTA or MDCT). The contrast media can be relatively viscous and often injected in a high flow, high speed manner (e.g. 3-5 cc/second). Thus, CT injections should be performed only through implanted ports rated for power injection.

Many vascular access ports are not power injectable rated or configured to withstand fluid flows greater than 1 cc/second or capable of accommodating a pressure within the reservoir up to about 300 psi. Users must be careful not to perform a high pressure power injection into an implantable port rated for lower pressures. Access ports that are not structured to withstand the pressures of a desired injection rate may cause a pressure within the system to exceed the pressure limit for components. Rupture can occur when the injection pressure exceeds the tolerance of the vascular access device. This can cause serious harm to patients.

Because the implanted port is positioned under the skin of the patient ascertaining a pressure rating of the port during and after use is difficult for medical personnel. Generally, medical personnel must depend upon reading a patient's chart to know the pressure rating of the implanted port and have no way to confirm the accuracy of the information recorded. There continues to be a need for medical personnel to identify the pressure rating of an implanted port in an easy manner that is accurate and that uses readily available equipment already widely available in hospitals and medical offices.

Described herein are vascular access port devices, systems, and methods of use. The vascular access ports described herein incorporate one or more markers configured to provide visual identification and verification of the pressure rating of the vascular access port as being non-power injection compatible under X-ray or fluoroscope. In some implementations, the marker identifies the vascular access port as a non-power injection vascular access port configured to accommodate lower fluid flow rates and structured to withstand lower pressures, for example, in the delivery of long-term therapies such as chemotherapy or other long-term medications, parenteral nutrition and the like. In other implementations, the marker identifies the vascular access port as a power injection vascular access port configured to accommodate high-pressure application of contrast mediums, for example, used in staging for examination in computed tomography.

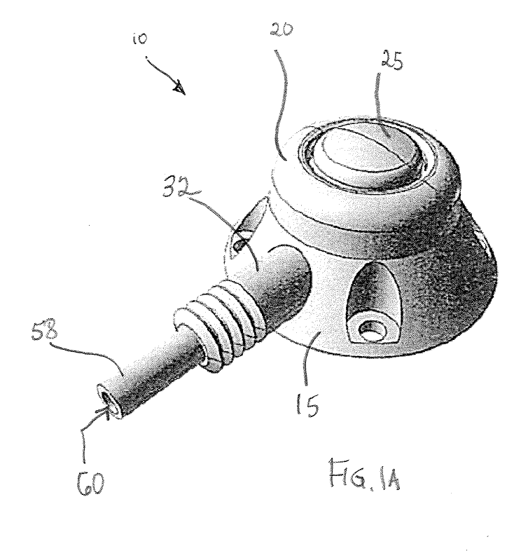

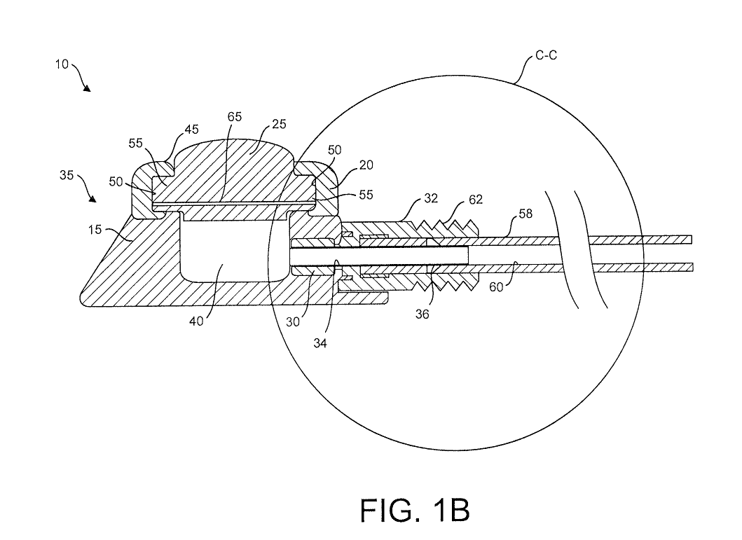

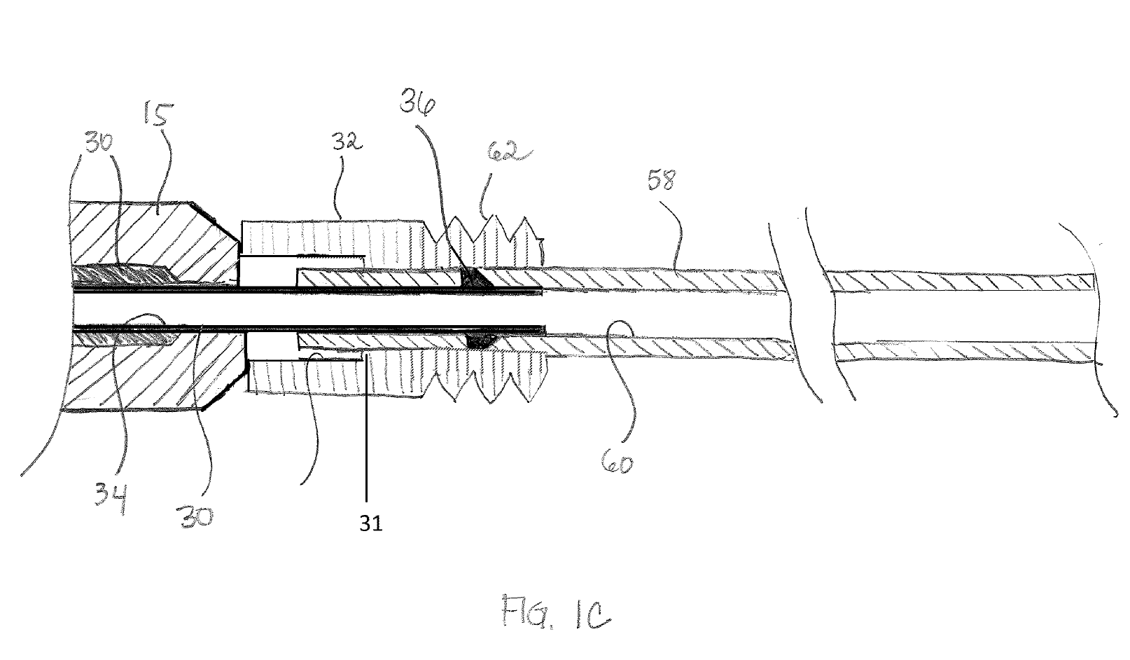

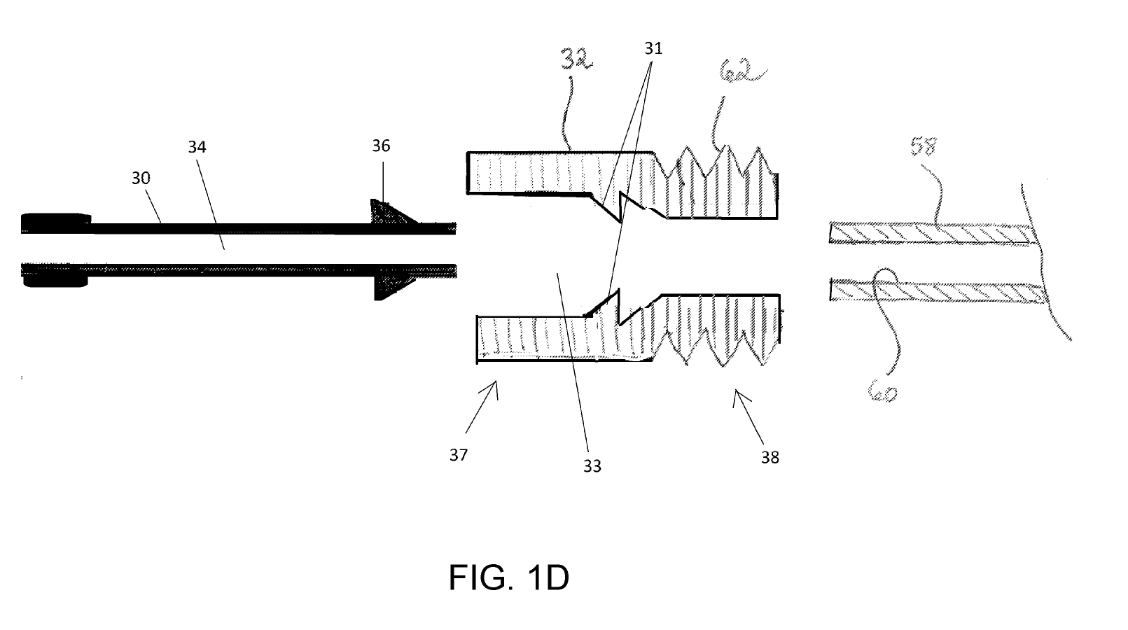

Referring now to the drawings, FIGS. 1A-1D show an implementation of a vascular access port 10. The port 10 includes a base 15, a cap 20, a septum 25, and an outlet stem 30. The septum 25 can be captured between the base 15 and the cap 20, which can collectively form a housing 35 for capturing the septum 25. The cap 20 can be generally annular in shape such that a central region of the septum 25 extends through a central aperture 45 in the cap 20. The cap 20 may also include an internal recess 50 shaped to accept at least a perimeter portion 55 of the septum 25. The perimeter portion 55 of the septum 25 can be received within internal recess 50 of the cap 20 such that the central region of the septum 25 extends through the aperture 45 of the cap 20. The base 15 and septum 25 collectively define an internal reservoir 40. The reservoir 40 is configured to be in fluid communication with a lumen 34 of the outlet stem 30, which in turn can be engageable to a catheter 58. It should be appreciated that the access port 10 can include a plurality of reservoirs 40 and a plurality of lumens 34 in the outlet stem 30 such that the port 10 can be used for the simultaneous administration of incompatible medications in a manner that allows for minimal mixing. It should also be appreciated that the port 10 need not include the outlet stem 30 and can instead include an opening extending through the base 15 of the housing 35 out from the reservoir 40 and in a manner configured to couple with a distal end of a catheter.

As mentioned, the outlet stem 30 can be engageable to a catheter 58 having a lumen 60 placed in sealed communication with a blood vessel of the patient. Thus, the outlet stem 30 creates a fluid communicative passageway extending from the reservoir 40 and through the outlet stem 30, catheter 58, and into the interior of the patient. The catheter 58 can be coupled to the outlet stem 30 for fluid communication with the internal reservoir 40 and for conducting fluid to a desired remote location from the internal cavity 50. The outlet stem 30 can be a tubular element having a first end coupled to the base 15 of the housing 35 and a second, opposite end extending out from the base 15. The lumen 34 of the outlet stem 30 is in fluid communication with the reservoir 40 at the first end of the outlet stem 30 and is in fluid communication with the lumen 60 of the catheter 58 at an opposite end. At least a portion of the outer surface of the outlet stem 30 can have a sealing retention feature 36 configured to engage with the lumen 60 of the catheter 58 for securement of the catheter 58 to the stem 30. The retention feature 36 can encircle the stem 30 and have an enlarged outer diameter compared to the outer diameter of the stem 30. In some implementations, the stem 30 is a metal tube and the retention feature 36 is frusto-conical shaped feature encircling the outer surface of the tube. The frusto-conical shape of the feature 36 eases insertion of the catheter 58 over the stem 30 when pushed in a first direction (i.e. towards the base 15) and restricts removal of the catheter 58 from the stem 30 when pulled in a second direction (i.e. away from the base 15). The retention feature 36 can have an outer diameter that is greater than the inner diameter of the catheter 58. However, due to the material properties of the catheter 58, which can be a flexible polymer, relative to the retention feature 36, which can be a rigid metal material, the catheter 58 can be forced over the sealing feature 36 such that the sealing feature 36 presses against the inner surface of the catheter lumen deforming or otherwise engaging the wall of the catheter 58 upon insertion of the catheter 58 onto the stem 30.

The outlet stem 30 can be secured to the end of the catheter 58 via a rigid connecting element 32 used to secure the end of the catheter 58 to the outlet stem 30 from the reservoir 40. The connecting element 32 can be a cylindrical member slideably and coaxially engaged upon the catheter 58 enhancing the coaxially frictional engagement of the catheter 58 to the outlet stem 30. As best shown in FIGS. 1B-1D, the connecting element 32 can be slideably engaged upon the outer surface of the catheter 58 by sliding the connecting element 32 over the catheter 58. The catheter 58, in turn, can be engaged around the outlet stem 30. A force imparted circumferentially to the catheter 58 by the connecting element 32 sandwiches the catheter 58 between the connecting element 32 and the outlet stem 30 over which it engages and thereby acts to further bias the catheter 58 against its contact with the outlet stem 30. This securely engages the catheter 58 to the exterior surface of the outlet stem 30 including at least the region where the sealing surface feature 36 is located. During assembly of the catheter 58 to the access port 10, an end of the catheter 58 is engaged to the outlet stem 30 as described above. The connecting element 32 can have a first end 37 at least a portion of which is configured to insert within or abut the base 15 and a second, opposite end 38 configured to extend away from the base 15. A lumen 33 extends through the cylindrical connecting element 32 from the first end 37 to the second end 38 such that the connecting element 32 can be slipped over the catheter 58 and slid down to where the catheter 58 is engaged with the outlet stem 30. The inner diameter of the connecting element 32 is sized larger than the outer diameter of the catheter 58 such that the connecting element 32 can be passed over the catheter 58 freely and loosely. However, the combined outer diameters of the catheter 58 positioned over the retention feature 36 of the stem 30 results in a snug fit with the inner diameter of the connecting element 32.

The inner diameter of the connecting element 32 can be non-uniform. For example, In some implementations, the inner diameter near the first end 37 can be larger than the inner diameter near the second end 38 such that a retention feature 31 is created (see FIGS. 1C and 1D). Alternatively, the retention feature 31 can be at least one ridge, flange, bump, protrusion, or other textured retention feature 31 on at least a portion of the inner lumen 33 of the connecting element 32. The retention feature 31 is sized to slide over the retention feature 36 of the stem 30 upon application of an amount of pushing force on the connecting element 32. Upon passing the connecting element 32 a distance over the stem 30 towards the housing base 15, retention feature 31 slides beyond retention feature 36 such that an audible and/or tactile “click” indicates the connecting element 32 is in its final, secured position relative to the stem 30 and catheter 58. The “click” can be a result of the retention feature 31 within the connecting element 32 snapping past the retention feature 36 on the stem 30 and the first end 37 of the connecting element 32 abutting the housing 15.

It should be appreciated that any of a number of connecting mechanisms can be incorporated to provide retention between the connecting element 32 and the stem 30. It should also be appreciated that although the retention features 31, 36 are shown as integral with the stem 30 or connecting element 32, respectively. The retention features 31, 36 can be separate components providing the retention desired. For example, the retention feature 36 can be a snap ring positioned within a groove of the connecting element 32 that can enlarge in circumference upon passing retention feature 36 through the bore of the connecting element 32 and snap back to a smaller circumference to retain the stem 30.

The connecting element 32 can have gripping features 62 on at least a portion of its outer surface to improve friction between a user's fingers and the catheter 58 such that the connecting element 32 can be more easily slid along the outer surface of the catheter 58 and over the sealing retention feature 36. For example, the second end 38 of the connecting element 32 configured to extend away from the housing 15 can be textured with the gripping features 62 to improve handling as the first end 37 of the connecting element 32 is pushed towards the base 15. The gripping features 62 can include one or more textures, indentations, recesses, ridges, flanges, wings, planar protrusions, or other engageable elements on the generally cylindrical outer surface of the connecting element 32 configured to improve friction and grip for a user. The connecting element 32 can be transparent, translucent, or opaque polymeric material that is relatively rigid compared to the catheter 58 material.

The access port 10 can be implanted within a patient such that is received within a prepared pocket under a patient's skin. Thus, the overall dimensions of the access port 10 or the housing 35 of the port 10 can be kept to a minimum such that it can be suitable for use in smaller patients or implanted as a peripheral port such as in the arm. The housing 35 of the port 10 may be generally oval, circular, or another geometric shape. The housing 35 of the port 10 can be formed of generally lightweight materials to prevent migration and/or discomfort to the patient. The access port 10 can be formed to have smooth edges and an ergonomic design to improve insertion into a patient. In some implementations, the access port 10 can include suture holes such that it can be sutured to affix the port 10 within the patient. In some implementations, the housing 35 of the port 10 can be formed of any of a variety of biocompatible materials, including, polysulfone, polyoxymethylene, titanium, or combinations thereof.

The base 15, cap 20 and septum 25 can be coupled together in any of a variety of ways including welding, brazing, soldering, fastening element, adhesive, or a combination thereof.

The upper surface of the septum 25 can be positioned such that upon implantation under the skin the upper surface of the septum 25 is aligned generally flush with the skin such that it may be repeatedly punctured for creating a percutaneous passageway from the exterior of the skin of the patient into the internal reservoir 40. The septum 25 is configured to be repeatedly pierced or punctured with a non-coring needle or other elongate element such as a Huber needle or cannula. The septum 25 can be formed of highly compressed silicone membrane for secure closing of septum and secure holding of puncturing needle. The septum can be easily palpable for safe identification of the puncture site. For example, the septum 25 can be slightly raised relative to the cap 20 surrounding the septum 25 such that the septum 25 can provide tactile feedback.

As fluid is injected into the reservoir 40 a positive pressure develops. The positive pressure within the access port 10 can act upon the septum 35 and the connection between the septum 35, the cap 20, and base 15 as well as the outlet stem 30 connection with the base 15 and/or the catheter 58. The septum 25, cap 20, and base 15 can withstand forces developed by the increase in pressure within the reservoir 40 without sustaining damage. In some implementations, the access port 10 is configured only for non-power injections such that it is configured to withstand fluid flow rates of no more than 1 mL/second through the port. In some implementations, the access port 10 is configured only for non-power injections just that it is configured to withstand a pressure within the reservoir 40 that is no more than about 35 psi at a temperature of 37° C. to 38° C. through the port without causing damage or compromising structural integrity of the reservoir, septum or another component of the port 10. In other implementations, the access port 10 is configured for either non-power or power injections such that it is configured to withstand fluid flow rates of up to 5 mL/second through the port. In some implementations, the access port 10 is configured for either non-power or power injections such that it is configured to withstand pressures within the reservoir 40 up to a maximum pressure of 300 psi at a temperature of 37° C. to 38° C. at a flow rate of about 5 ml/second through the port without causing damage or compromising structural integrity of the reservoir 40, septum 25, or other component of the port 10.

Access ports suitable for power injections must meet a higher standard in testing and validation compared to access ports suitable for non-power injections. Due to the stringent testing during manufacturing, these ports tend to have a higher price differential in the marketplace. Thus, there is a need for providing non-power injection rated ports for certain indications where high fluid flow rates are unnecessary. However, there is also a need for easily identifying non-power injection ports such that high fluid flow rates are not inadvertently performed on a port not rated for such injections.

In some implementations, the perimeter portion 55 of the septum 25 configured to couple with an internal recess 50 of the cap 20 can provide for improved mechanical constraint under these higher pressure ranges. It should be appreciated that any number of various coupling features can be incorporated to ensure the septum 25 is mechanically secured to the housing 25. For example, the septum 25 can be coupled to the housing 25 with complementary coupling features including one or more ribs, flanges, interlocking features, tenon and mortise type features, tongue-in-groove features, t-slot, dovetail, snap-fit, tabs, slots and other coupling features. These couplings features allow for the access port to be used for infusing fluids at higher flow rates and higher internal pressures without compromising structural integrity of the port 10. The access port 10 configured for power injection can also incorporate one or more structural elements configured to support or supplement the mechanical coupling of the septum 25 to the housing 35. The structural elements can have any of a variety of configurations including a wire, pin, columnar element, filament and can be formed of any of a variety of materials including titanium, stainless steel, polymer, or other biocompatible material or composite configured to resist deformation of the structural components of the port 10, particularly the septum 25.

As will be described in more detail below, the access ports described herein can include one or more markers 65 discernable following subcutaneous implantation of the access port in a patient and configured to identify whether the port is suitable for or compatible with power injection fluid flow rates or non-power injection fluid flow rates. The marker 65 can be formed of one or more materials that are easily discerned on a CT scan or X-ray or on fluoroscope relative to the surrounding tissues as well as relative to the other material components of the port 10 itself. For example, the material of the marker 65 can be one or more of nitinol, tungsten, titanium, stainless steel, aluminum, copper, tin, nickel, or other non-ferrous metal. The marker 65 can be formed of an X-ray discernable material such as high density ceramic, gadolinium oxysulfide, silicone nitride, zirconium and zirconium oxide, or X-ray excitable polymers such as PTFE or PTFE impregnated with a non-ferrous metal. The marker 65 can be formed of inks formed of a biocompatible carrier containing one or a combination of the x-ray discernable materials. The inks may be printed or adhered to a surface of the port 10 and can provide contrast with surrounding tissues and materials forming other components of the port 10. It should be appreciated that the marker 65 can be MRI-safe and formed substantially of non-ferrous metal that would not be moved or dismounted or attracted to the magnetic forces of an MRI or be substantially heated. The marker 65 can be positioned such that the marker avoids being scraped off or otherwise damaged during implantation or removal from a patient. In some implementations, the marker 65 can but need not be engaged to the port 10 using adhesive or heating or other engagement method to a surface of the port 10.

The one or more markers 65 can be structural elements coupled to one or more regions of the access port. The one or more markers 65 can be two-dimensional or three-dimensional. The one or more markers 65 can be elongated elements such as a wire, ribbon, thread, fiber, or columnar element. The one or more markers 65 can be a mesh, fabric, coating, or other generally planar element. The one or more markers 65 can be arranged in the shape of non-alphanumeric symbols and/or shapes that may be understood regardless of the language spoken by the reader. The one or more markers 65 can be formed from a solid piece of metal material in a non-letter symbol. The size of the marker 65 can be suitable for the visually impaired and need not require a user to read words. The one or more markers 65 can be penetrated such as by a cannula or syringe needle inserted through the septum 25. The one or more markers 65 can be a geometric shape such as a circle, ellipse, triangle, rectangle, etc.

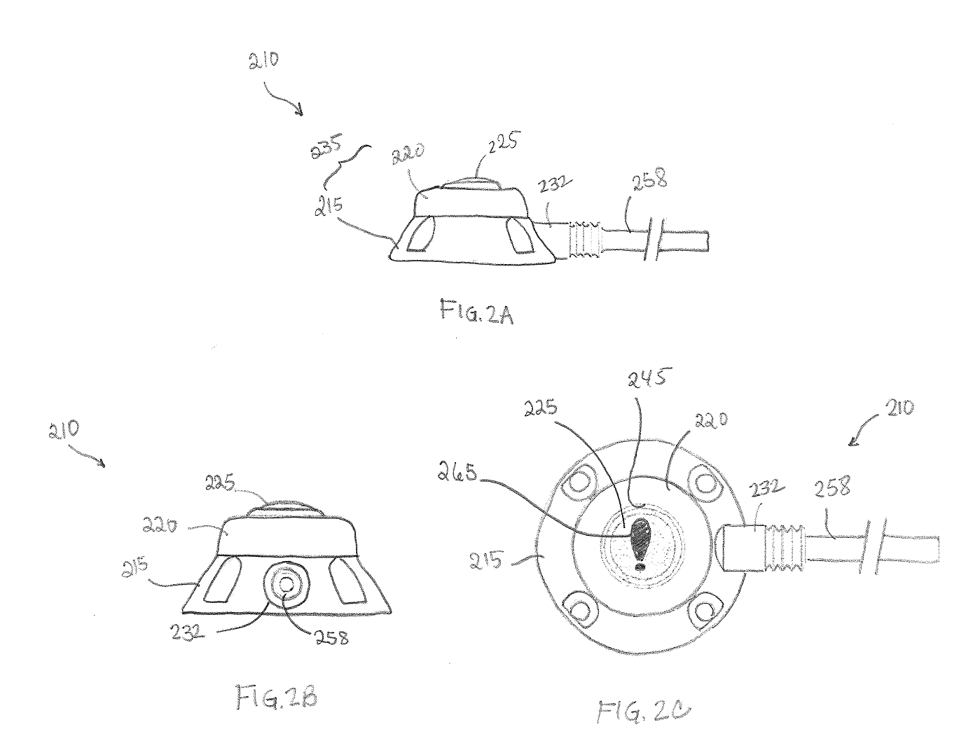

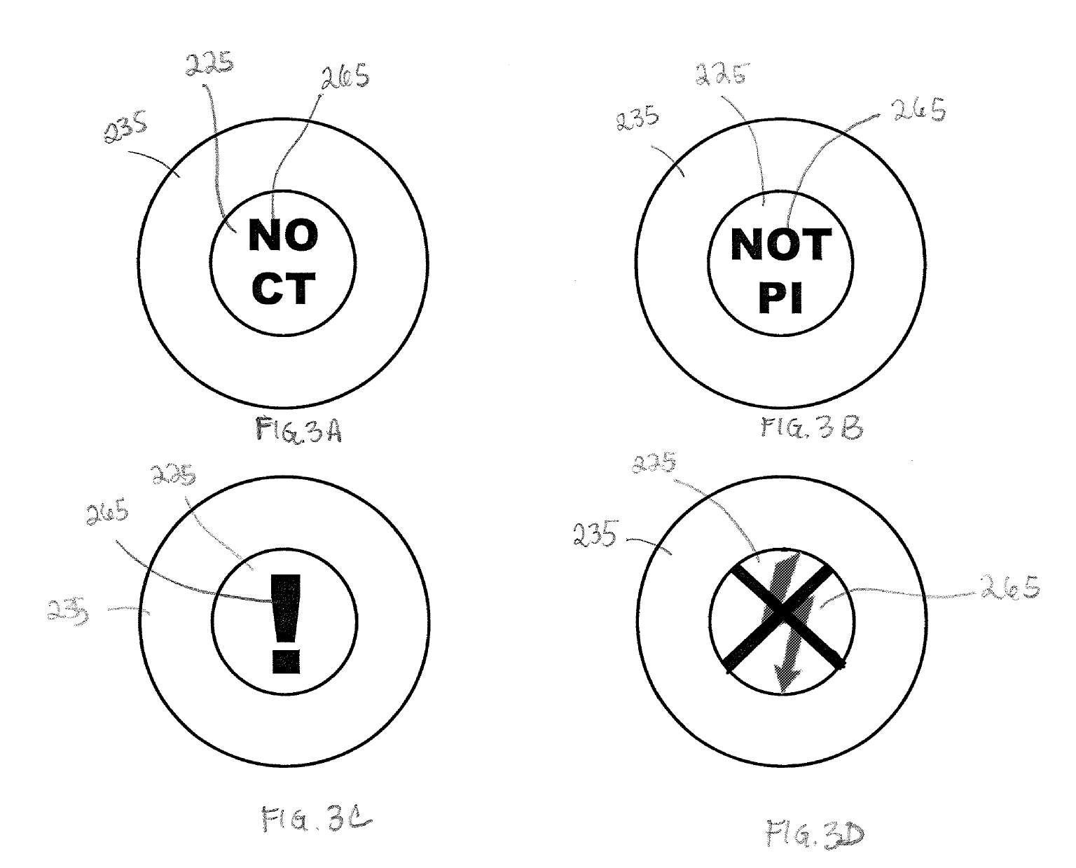

FIGS. 2A-2C show an implementation of an access port 210 having a marker 265 that identifies the port 210 as being configured for non-power injections such as for the delivery of long-term medications, non-parenteral nutrition, or other purposes that do not require high pressures and high fluid flow rate injections and the one or more markers 265 identify the access port 210 as such. As described above, the access port 210 includes a base 215, a cap 220, a septum 225, and an outlet stem (not visible) coupled to a connecting element 232 positioned on an end of a catheter 258. The septum 225 can be captured between the base 215 and the cap 220, which can collectively form a housing 235 for capturing the septum 225. The cap 220 can be generally annular in shape such that a central region of the septum 225 extends through a central aperture 245 in the cap 220. The base 215 and septum 225 collectively define an internal reservoir (not visible) configured to be in fluid communication with a lumen of the outlet stem, which in turn can be engageable to a catheter 258. The one or more markers 265 can be positioned on and/or in the housing 235, the septum 225, or a combination thereof In some implementations, the marker 265 is positioned within the interior of the reservoir. In other implementations, the marker 265 is embedded in or attached to a surface of the septum 225. In other implementations, the marker 265 is positioned on a region of the housing 235, such as on a bottom surface of the base 215, a side of the base 215, a surface of the cap 220, or on the connecting element 232.

In some implementations, as shown in FIGS. 3A-3B, the non-power injection port marker 265 can be an alphanumeric symbol, word or phrase such as “No CT” or “NOT PI” or “No HP” or “Not HP” or “Not For Power Injection” or “Non Power” other type of message that identifies the port as not be configured for high pressure and/or high flow rate injections. The exact pressure rating of the port can also be embedded on or within the septum 225 such that the flow rate or pressure range is specified and visible under X-ray, CT, or fluoroscope. In other implementations, the non-power injection port marker 265 can be a non-alphanumeric symbol such as an exclamation point or other shape or symbol identifying the port as not being configured for high pressure injections (see FIGS. 3C-3D). In some implementations, the non-power injection port marker 265 can be a shape or symbol indicative of a power injection port, such as a triangle or a lightning bolt, but having an X extending through it as shown in FIG. 3D.

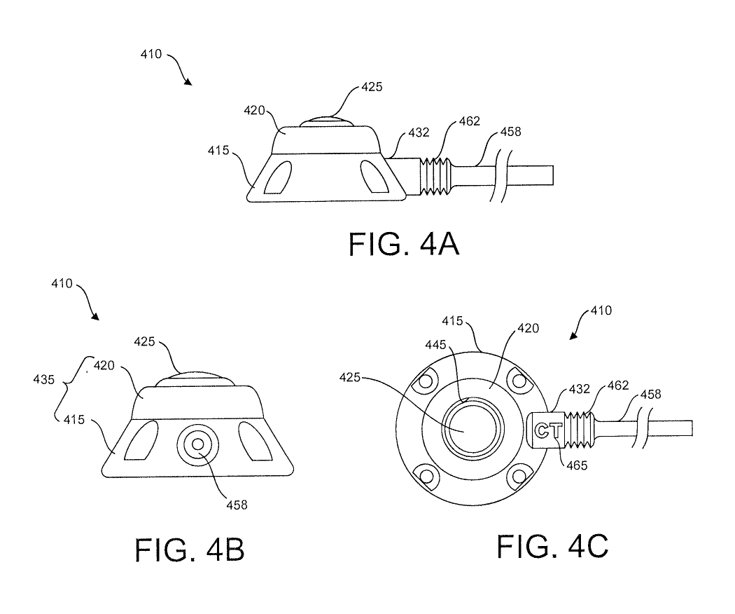

In some implementations, the one or more markers identify the port as being compatible for power injection or non-power injection can be positioned on the connecting element. FIGS. 4A-4C show an implementation of an access port 410 having a marker 465 that identifies the port 410 as being configured for power injections (or non-power injections) and incorporating a marker 465 on the connecting element 432. As described above, the access port 410 can include a base 415, a cap 420, a septum 425, and an outlet stem (not visible). The septum 425 can be captured between the base 415 and the cap 420, which can collectively form a housing 435 for capturing the septum 425. The cap 420 can be generally annular in shape such that a central region of the septum 425 extends through a central aperture 445 in the cap 420. The base 415 and septum 425 collectively define an internal reservoir (not visible) configured to be in fluid communication with a lumen of the outlet stem, which in turn can be engageable to a catheter 458. The one or more markers 465 can be positioned on the connecting element 432. As described elsewhere herein, the connecting element 432 can be a relatively rigid, cylindrical component configured to secure an end of the catheter 458 to the outlet stem of the access port such that the lumen of the catheter 458 is placed in fluid communication with the reservoir. The connecting element 432 can be slideably and coaxially engaged upon the catheter 458 and configured to enhance the coaxial frictional engagement of the catheter 458 to the stem. The connecting element 432 can have a first end 464 configured to be located nearest the housing 435 upon connection to the access port 410 and an end configured to be handled by a user. At least a portion of the connecting element 432 can have gripping features 462 on its outer surface to improve handling. The first end 464 of the connecting element 432 can be devoid of the gripping features 462 such that the first end 464 can have the one or more markers 465 positioned on it.

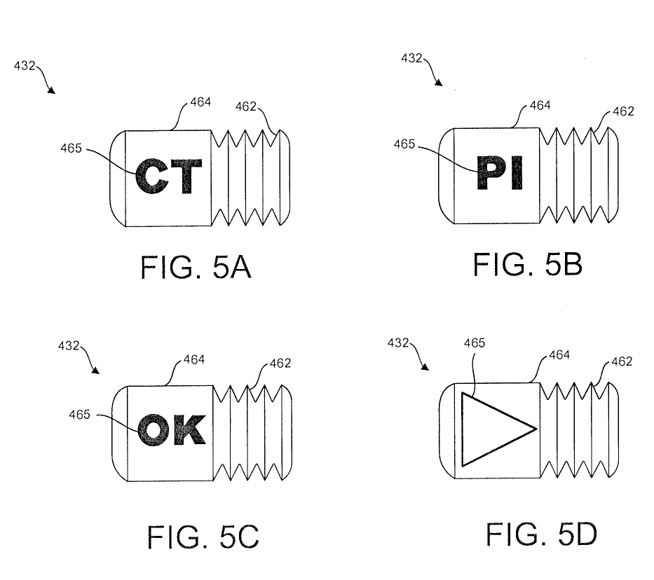

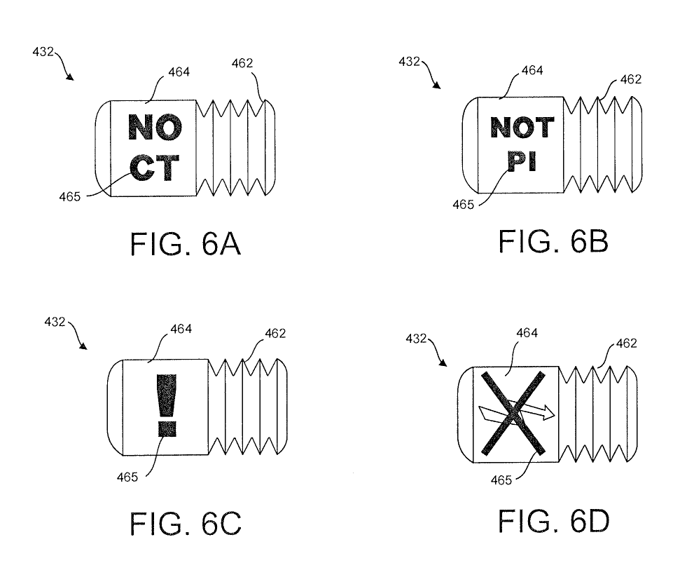

In some implementations as shown in FIGS. 5A-5C, the port marker 465 can be an alphanumeric symbol, word or phrases such as “CT” or “PI” or “HP” or “OK” or “For Power Injection” or “Power” or other type of message that identifies the port as being configured for high pressure and/or high flow rate. The exact pressure rating of the port can also be visually displayed on the connecting element 432 such that the flow rate or pressure range is specified and visible under X-ray, CT, or fluoroscope. In other implementations, the port marker 465 can be a non-alphanumeric symbol such as a triangle, check-mark, lightning bolt, or symbol identifying the port as being configured for high pressure injections (see FIG. 5D). The still other implementations, the port marker 465 can be an alphanumeric symbol, word, phrase, or a non-alphanumeric symbol identifying the port as being configured for non-power injections such as “NO CT” or “NOT PI” or “NO HP” or “Not For Power Injection” or “Non Power” other type of message that identifies the port as not be configured for high pressure (FIGS. 6A-6B). The exact pressure rating of the port can also be displayed on the connecting element 432 such that the flow rate or pressure range is specified and visible under X-ray, CT, or fluoroscope. In other implementations, the non-power injection port marker 465 can be a non-alphanumeric symbol such as an exclamation point or symbol identifying the port as not being configured for high pressure injections (see FIGS. 6C-6D).

During a CT scan, which concurrently requires the injection of a large volume of liquid by a power injection under high pressure, medical professional can easily ascertain whether the implanted access port has a high pressure rating required for the procedure or a low pressure rating. The medical professional can do so by taking a quick X-ray or the patient in the vicinity of the implanted access port. Because the access port is not pressure rated for the procedure, the marker will indicate to the medical professional the power injection should not be performed through this access port.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation,” “an implementation,” or the like, means that a particular feature, structure, configuration, or characteristic described is included in at least one embodiment or implementation. Thus, the appearance of the phrase “one embodiment,” “an embodiment,” “one implementation,” “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. However, such terms are provided to establish relative frames of reference, and are not intended to limit the use or orientation of the systems to a specific configuration described in the various implementations.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

What is claimed is:

1. A non-power injectable vascular access port configured to be implanted subcutaneously comprising:
a housing;
a septum affixed to the housing;
an internal reservoir collectively defined by the septum and the housing;
an outlet configured to be in fluid communication with the internal reservoir; and
one or more markers discernable following subcutaneous implantation of the access port and configured to identify the vascular access port as suitable for a non-power injection fluid flow rate and unsuitable for a power injection fluid flow rate.

2. The non-power injectable vascular access port of claim 1, wherein the non-power injection fluid flow rate is no more than about 1 mL/second through the port.

3. The non-power injectable vascular access port of claim 1, wherein the port is configured to withstand an internal pressure of no more than about 35 psi at a temperature of about 37° C.

4. The non-power injectable vascular access port of claim 1, wherein the one or more markers is formed of a material that is discernable on a CT scan, X-ray, or fluoroscope relative to surrounding tissues as well as materials of the port.

5. The non-power injectable vascular access port of claim 4, wherein the material is selected from one or more of the group consisting of nitinol, tungsten, titanium, stainless steel, aluminum, copper, tin, nickel, non-ferrous metal, high density ceramic, gadolinium oxysulfide, silicone nitride, zirconium, zirconium oxide, X-ray excitable polymer, PTFE, and PTFE impregnated with a non-ferrous metal.

6. The non-power injectable vascular access port of claim 1, wherein the one or more markers is an ink printed on a surface of the port.

7. The non-power injectable vascular access port of claim 1, wherein the one or more markers is a structural element coupled to one or more regions of the access port.

8. The non-power injectable vascular access port of claim 1, wherein the one or more markers is two-dimensional or three-dimensional.

9. The non-power injectable vascular access port of claim 1, wherein the one or more markers is an elongated wire, ribbon, thread, fiber, or columnar element.

10. The non-power injectable vascular access port of claim 1, wherein the one or more markers is a mesh, fabric, coating, or other generally planar element.

11. The non-power injectable vascular access port of claim 1, wherein the one or more markers is formed from a solid piece of metal material.

12. The non-power injectable vascular access port of claim 1, wherein the one or more markers is penetrable by a cannula or syringe needle inserted through the septum.

13. The non-power injectable vascular access port of claim 1, wherein the one or more markers is arranged in the shape of a non-alphanumeric symbol and/or shape.

14. The non-power injectable vascular access port of claim 1, wherein the one or more markers is a non-letter symbol.

15. The non-power injectable vascular access port of claim 14, wherein the non-letter symbol is selected from the group consisting of an exclamation point, a lightning bolt with an X through it, and a triangle with an X through it.

16. The non-power injectable vascular access port of claim 1, wherein the one or more markers is an alphanumeric symbol, word or phrase.
